(19) United States
(12) Patent Application Publication
(10) Pub. No.: US 2024/0277862 A1
(43) Pub. Date: Aug. 22, 2024

(54) BISPECIFIC ANTIBODIES USING FUNCTIONALIZED POLY-ADP-RIBOSE POLYMERS

(71) Applicant: UNIVERSITY OF SOUTHERN CALIFORNIA, Los Angeles, CA (US)

(72) Inventor: Yong ZHANG, Los Angeles, CA (US)

(21) Appl. No.: 18/439,252

(22) Filed: Feb. 12, 2024

Related U.S. Application Data
(60) Provisional application No. 63/484,707, filed on Feb. 13, 2023.

(51) Int. Cl. A61K 47/68; A61K 38/45; A61P 35/00; C07K 16/28; C07K 16/32

(52) U.S. Cl. CPC A61K 47/6815; A61K 38/45; A61K 47/6849; A61K 47/6889; A61P 35/00; C07K 16/2809; C07K 16/32; C12Y 204/02039

(57) ABSTRACT

A poly ADP-ribose polymerase (PARP)-antibody conjugate including an automodified PARP having a plurality of poly ADP-ribose (ADPr) polymers, wherein the poly ADPr polymers comprise a plurality of 3′-azido ADP-ribose moieties; and one or more antibody molecules conjugated to one or more of the plurality of 3′-azido ADP-ribose moieties, wherein the one or more antibody molecules specifically bind to both a cancer cell surface marker protein and an immune cell surface marker protein, and wherein at least one of the plurality of 3′-azido ADP-ribose moieties is not conjugated to the antibody molecules.

### RELATED APPLICATIONS

This application claims priority under 35 U.S.C. § 119(e) to U.S. Provisional Patent Application No. 63/484,707, filed Feb. 13, 2023, which is incorporated herein by reference.

GOVERNMENT SUPPORT

This invention was made with government support under grant no. R35GM137901, awarded by the (NIH/NIGMS) National Institute of General Medical Sciences and grant no. R01EB031830, awarded by the (NIH/NBIB) National Institute of Biomedical Imaging and Bioengineering. The government has certain rights in the invention.

### BACKGROUND OF THE INVENTION

Protein poly-ADP-ribosylation (PARylation) occurs through covalent attachments of ADP-ribose moieties from nicotinamide adenine dinucleotide (NAD⁺) onto target proteins. This post-translationally modifying process is catalyzed by endogenous poly(ADP-ribose) polymerases (PARPs) and features formation of linear and branched ADP-ribose polymers with varied sizes and patterns. The resulting heterogeneous ADP-ribose polymers are involved in regulating DNA repair and many other cellular activities. Despite extensive studies of PARylation in physiology and pathophysiology, the potential of the ADP-ribose polymers for therapeutic applications remains untapped. As a natural form of polymer, poly-ADP-ribose (PAR) is characterized by high hydrophilicity and biocompatibility and may therefore serve as promising drug carriers. Accordingly, there is a need for clinically effective targeted compounds and methods of use of such compounds to treat various disorders, such as cancers. The present disclosure satisfies these needs.

### SUMMARY OF THE INVENTION

Embodiments of the invention are directed to methods, compounds, antibody-drug conjugates, and pharmaceutical compositions containing antibody-drug conjugates (ADCs), the ADCs comprising an auto-modified poly ADP-ribose polymerase (PARP) as a novel drug carrier. Due to robust automodification activity, human PARP may catalyze the transfer of Adenine Diphosphate-ribose (ADPr) (also known as PARylation) with clickable moieties onto itself, resulting in a plurality of clickable ADPr polymers on the surface of the PARP. The generated automodified PARP with clickable polymers allows conjugation with antibodies, antibody fragments, or other binding proteins, and with cytotoxic agents, to form novel ADCs. The generated ADCs display significant specificity and potency toward targeted cell lines. This versatile platform provides a new class of ADCs with improved efficacy.

The present disclosure investigates whether functionalized PAR polymers conjugated to two or more antibodies, or bispecific antibodies may be used for cancer immunotherapy. By simultaneously engaging both tumors and immune effector cells, genetically or chemically engineered bispecific antibodies promote formation of immunological synapses and induction of tumor-specific immunity, establishing new molecular therapeutics for malignancies. To test this notion, azido-functionalized PAR polymers were utilized to conjugate with both anti-human CD3 and anti-human epidermal growth factor receptor 2 (HER2) antibodies (FIG. 1). The resulting PAR-antibody conjugate shows potent HER2-dependent cytotoxicity in the presence of non-activated human peripheral blood mononuclear cells (PBMCs), demonstrating a new approach for synthesizing bispecific antibodies.

In some embodiments, a poly ADP-ribose polymerase (PARP)-antibody conjugate comprises an automodified PARP comprising a plurality of poly ADP-ribose (ADPr) polymers, wherein the poly ADPr polymers comprise a plurality of 3′-azido ADP-ribose moieties; and one or more antibody molecules conjugated to one or more of the plurality of 3′-azido ADP-ribose moieties, wherein the one or more antibody molecules specifically bind to both a cancer cell surface marker protein and an immune cell surface marker protein, and wherein at least one of the plurality of 3′-azido ADP-ribose moieties is not conjugated to the antibody molecules.

In some embodiments, the plurality of 3′-azido ADP-ribose moieties have a structure according to Formula I:

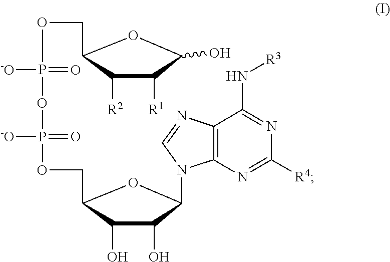

wherein R¹ is OH, R² is N₃, R³ is H, and R4 is H; or a partially or fully protonated structure thereof; or a salt (e.g., a sodium salt) thereof; and wherein conjugation can occur via the azide of R² and/or via the anomeric position of the terminal sugar moiety of Formula I (which result in the corresponding modifications of Formula I as a conjugated moiety, as described herein). For example, when a 3′-azido ADP-ribose moiety is conjugated to PARP1, a nucleotide of a different 3′-azido ADP-ribose moiety, or an antibody, the corresponding structure of Formula I can be:

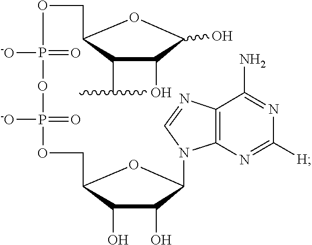

or a partially or fully protonated structure thereof; or a salt (e.g., a sodium salt) thereof, wherein the conjugation occurs via the site formerly occupied by the azide moiety, which is transformed into a ‘click chemistry’ linker.

In some embodiments, the automodified PARP (i.e, via the 3′azido ADP-ribose comprising the ADP-rose polymers) is linked to the antibody through an alkyne-derived linking group or a polyethylene glycol linking group. In other embodiments, the modified PARP is linked to the antibody through an alkyne-derived linking group, a cyclooctyne-derived linking group, or a polyethylene glycol linking group. In some embodiments, the alkyne-derived linking group is an alkyne-PEG4-NHS ester linking group.

In some embodiments, the PARP is PARP1, PARP2, PARP5a, or PARP5b.

In some embodiments, the antibody molecule comprises a first antibody and a second antibody, wherein the first antibody specifically binds to a cancer cell surface marker protein and the second antibody specifically binds to an immune cell surface marker protein.

In some embodiments, the antibody molecule comprises a bispecific antibody, wherein the bispecific antibody comprises a first antibody moiety and a second antibody moiety, wherein the first antibody moiety specifically binds to a cancer cell surface marker protein and the second antibody moiety specifically binds to an immune cell surface marker protein.

In some embodiments, the immune cell surface marker protein comprises one or more of CD3, OX40, CD2, CD4, CD5, CD7, CD8, CD14, CD15, CD16, CD24, CD25, CD27, CD28, CD30, CD31, CD38, CD40L, CD45, CD56, CD68, CD91, CD114, CD163, CD206, LFA1, PD-1, ICOS, BTLA, KIR, CD137, LAG3, CTLA4, or a T-cell Receptor.

In some embodiments, the cancer cell surface-marker protein comprises one or more of EGFR, CLL-1, HER2, HER3, CD33, CD34, CD38, CD123, TIM3, CD25, CD32, CD96, PD-L1, and PD-L2. In some embodiments, the cancer cell surface-marker protein is HER2 and the immune cell surface-marker protein is CD3.

In some embodiments the antibody molecule comprises a monoclonal antibody, polyclonal antibody, a single chain Fv, a bispecific antibody, a multispecific antibody, a Fv fragment, a Fab fragment, and a F(ab)₂ fragment.

The disclosure also provides for methods of preparing a poly ADP-ribose polymerase (PARP)-antibody conjugate comprising the steps of combining a linker and an antibody molecule to provide an antibody-linker conjugate; combining a PARP and a plurality of an azido substituted dinucleotide to provide an automodified PARP comprising a plurality of poly ADP-ribose (ADPr) groups on a surface of the automodified PARP; and combining the automodified PARP and the antibody-linker conjugate under suitable conditions such that the antibody-linker conjugate is conjugated to the azido substituted dinucleotide through click chemistry to form the PARP-antibody conjugate. In some embodiments, the azido substituted dinucleotide is 3′-azido NAD⁺. In some embodiments, the plurality of poly ADP-ribose (ADPr) groups on a surface of the automodified PARP comprise 3′-azido ADP-ribose.

The present invention and its attributes and advantages will be further understood and appreciated with reference to the detailed description below of presently contemplated embodiments, taken in conjunction with the accompanying drawings.

### BRIEF DESCRIPTION OF THE DRAWINGS

The following drawings form part of the specification and are included to further demonstrate certain embodiments or various aspects of the invention. In some instances, embodiments can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1 illustrates a schematic of the design and generation of a PAR polymer-based bispecific antibody conjugate.

FIG. 2A-D illustrates the generation and characterization of the PARylated PARP1-Herceptin/UCHT1 conjugate. (A) Coomassie stain of purified human PARP1, Herceptin, and UCHT1 antibody. (B) PARylated PARP1 by 3′-azido NAD³⁰ as revealed through immunoblotting using an anti-PAR antibody. (C) Conjugation of Herceptin and UCHT1 antibody with PARylated PARP1 by 3′-azido NAD⁺ as revealed by Coomassie stain (left) and immunoblotting (right) using an anti-human IgG antibody-HRP. (D) Flow cytometric analysis of the binding of fluorescein-labeled PARylated PARP1-Herceptin/UCHT1 conjugate to HCC 1954 cells (HER2⁺), Jurkat cells (CD3⁺), and MDA-MB-468 cells (HER2⁻CD3⁻). Fluorescein-labeled PARylated PARP1 was used as a control.

FIG. 3A-E illustrates in vitro cytotoxicity of the PARylated PARP1 -Herceptin/UCHTI conjugate. Human PBMCs (effector cells) were incubated with breast cancer cells (target cells) at an E:T ratio of 10 in the presence of various concentrations of the PARylated PARP1-Herceptin/UCHTI conjugate. PARylated PARP1 was used as a control. (A) SK-BR-3 (Her2+ cells); (B) HCC 1954 Her2+ cells), (C) MCF-7 (Her2+ cells); (D) MDA-MB-231 (Her2+ cells): (E) MDA-MB-468 (Her2− cells). Data are shown as mean ±SD of triplicates.

### DETAILED DESCRIPTION

The present disclosure is directed to PARP-antibody conjugates, compositions comprising the PARP-antibody conjugates, and methods for preparing the PARP-antibody conjugates. Generally, a PARP-antibody conjugate comprises an automodified PARP comprising a plurality of poly ADP-ribose (ADPr) polymers on a surface of the automodified PARP, wherein the poly ADPr polymers comprise one or more 3′-azido NAD³ moieties; and an antibody molecule conjugated to at least one of the one or more 3′-azido NAD³ moieties of the poly ADPr polymers. The antibody molecule may comprise a first antibody and a second antibody that specifically binds to different antigens. In other embodiments, the antibody molecule may be a bispecific antibody (i.e., an antibody that binds to two separate antigens).

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991). General laboratory techniques (DNA extraction, RNA extraction, cloning, cell culturing. etc.) are known in the art and described, for example, in Molecular Cloning: A Laboratory Manual, J. Sambrook et al., 4th edition, Cold Spring Harbor Laboratory Press, 2012.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The term “about” can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, composition, or embodiment. The term about can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect. An appropriate “effective” amount in any individual case may be determined using techniques, such as a dose escalation study.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods provided herein, the mammal is a human.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “antibody” as used herein refers to a polypeptide (or set of polypeptides) of the immunoglobulin family that is capable of binding an antigen non- covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VII) and a heavy chain constant region. The heavy chain constant region is comprised of three domains, CHI, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FRI, CDR1, FR2, CDR2, FR3, CDR3, FR4. The variable regions of the heavy and light chains contain a binding domain that interacts with an antigen, which is sometimes referred to herein as the antigen binding domain. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelised antibodies, chimeric antibodies, bispecific or multispecific antibodies and anti-idiotypic (anti-Id) antibodies (including, e.g., anti-Id antibodies to antibodies described herein), single chain variable fragments, and single domain antibodies. The antibodies can be of any isotype/class (e.g., IgG, IgE, IgM, IgD, IgA and IgY) or subclass (e.g., IgGI, IgG2, IgG3, IgG4, IgAl and IgA2). Both the light and heavy chains are divided into regions of structural and functional homology. The terms “constant” and “variable” are used functionally. In this regard, it will be appreciated that the variable domains of both the light (V_{L}) and heavy (V_{H}) chain portions determine antigen recognition and specificity. Conversely, the constant domains of the light chain (CL) and the heavy chain (CHI, CH2 or CH3) confer important biological properties such as secretion, transplacental mobility, Fc receptor binding, complement binding, and the like. By convention the numbering of the constant region domains increases as they become more distal from the antigen binding site or amino- terminus of the antibody. The N-terminus is a variable region and at the C-terminus is a constant region; the CH3 and CL domains actually comprise the carboxy-terminus of the heavy and light chain, respectively.

As used herein, “fragment” is defined as at least a portion of the variable region of the immunoglobulin molecule which binds to its target, i.e., the antigen binding region. Some of the constant region of the immunoglobulin may be included.

As used herein, “fused” means to couple directly or indirectly one molecule with another by whatever means, e.g., by covalent bonding, by non-covalent bonding, by ionic bonding, or by non-ionic bonding. Covalent bonding includes bonding by various linkers such as thioether linkers or thioester linkers. Direct fusion involves one molecule attached to the molecule of interest. Indirect fusion involves one molecule attached to another molecule which in turn is attached directly or indirectly to the molecule of interest.

As used herein, the terms “peptide,” “polypeptide,” and “protein” are used interchangeably, and refer to a compound comprising amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein, is meant a molecule, such as an antibody, which recognizes and binds to another molecule or feature, but does not substantially recognize or bind other molecules or features in a sample.

“Poly adenosine diphosphate ribose (ADP) transferase activity” intends the activity of Poly-(ADP-ribose) polymerases (PARPs) that are found mostly in eukaryotes and catalyze the transfer of multiple ADP-ribose molecules to target proteins. As with mono-ADP ribosylation, the source of ADP-ribose is NAD⁺. PARPs use a catalytic triad of His-Tyr-Glu to facilitate binding of NAD and positioning of the end of the existing poly-ADP ribose chain on the target protein; the Glu facilitates catalysis and formation of a (1−>2) O-glycosidic linkage between two ribose molecules. There are several other enzymes that recognize poly-ADP ribose chains, hydrolyze them or form branches.

As used herein, the term “ADP-ribose unit” intends a compound comprising, consisting essentially of, or yet further consisting of an adenosine and a ribose joined through a diphosphate group.

“Adenosine diphosphate ribose (ADP) ribosyltransferase activity” intends the intracellular action of the addition of one or more ADP-ribose moieties to a protein. It is a reversible post-translational modification that is involved in many cellular processes, including cell signaling, DNA repair, gene regulation and apoptosis. Improper ADP-ribosylation has been implicated in some forms of cancer.

“Nicotinamide adenine dinucleotide” (NAD) (also known as diphosphopyridine nucleotide (DPN+) and Coenzyme I) refers to the coenzyme that contains an adenine nucleobase and a nicotinamide nucleobase, which coenzyme is found in all cells. The compound is a dinucleotide consisting of two nucleotides joined through their phosphate groups. NAD exists in an oxidized or reduced form, which are abbreviated as NAD and NADH (H for hydrogen), respectively. The chemical structure of NAD can be protonated or found in its salt form, for example, its disodium salt.

### PARPs.

Poly-ADP-ribose polymerases (PARPs), also known as ADP-ribosyltransferases (ARTs), are emerging as major effectors of NAD -mediated signaling in cells. PARPS are a diverse family of at least 17 mammalian enzymes that catalyze the reversible post-translational modification—known as ADP-ribosylation, or PARylation—involving the transfer of ADP-ribose from NAD to target proteins. This enzymatic posttranslational modification requires nicotinamide adenine dinucleotide (NAD) as a donor of ADP-ribose (i.e., the NAD⁺ is lost during formation of the ADP polymers). Upon covalent attachments of ADP-ribose to side chains of various types of amino acid residues, PARPs may continue adding ADP-ribose sequentially at ribosyl 2′-OH positions, resulting in linear or branched poly-ADP-ribose (PAR) polymers with up to 300 ADP-ribose units in length. Exemplary PARPS include, but are not limited to, PARP1, PARP2, PARP5α, and PARP5β.

### Nicotinamide Adenine Dinucleotide Analogue.

In various embodiments, the nicotinamide adenine dinucleotide (NAD⁺) that acts a donor of ADP-ribose in the automodification and/or PARylation reaction is modified at the 2′-OH (R¹ position), the 3′-OH (R² position) position, the 6-amine position of the purine moiety (R³ position), and/or the 2-position of the purine moiety (R⁴ position) and may include a chemical group that may undergo “click chemistry”. Various embodiments include a modified NAD⁺ analogue having a “click chemistry” group according to Table 1 below.

TABLE 1
Chemical structures of NAD⁺ analogues 1-9.

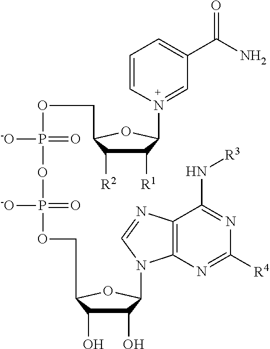

| | | | | |
|---|---|---|---|---|
| 1: | 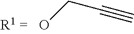 | R² = OH | R³ = H | R⁴ = H |
| 2: | R¹ = OH | 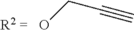 | R³ = H | R⁴ = H |
| 3: | 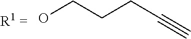 | R² = OH | R³ = H | R⁴ = H |
| 4: | R¹ = OH | 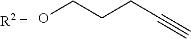 | R³ = H | R⁴ = H |
| 5: | R¹ = N₃ | R² = OH | R³ = H | R⁴ = H |
| 6: | R¹ = OH | R² = N₃ | R³ = H | R⁴ = H |
| 7: | 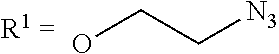 | R² = OH | R³ = H | R⁴ = H |
| 8: | R¹ = OH | R² = OH | 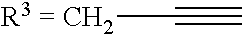 | R⁴ = H |
| 9: | R¹ = OH | R² = OH | R³ = H | 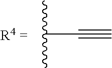 [SMILES: *C#C] |

Various modified NAD⁺ analogue embodiments include modified NAD⁺ analogues having an alkyne or azido group, such as those shown above in Table 1, at any of the R¹-R⁴ positions, in any different combination. For example, any of analogues 1-7 may also be substituted with an alkyne at the R³ or R⁴ positions, and of analogues 8-9 can be substituted at the R¹ or R² positions with an oxygen-linked alkyne or alkyl azido group, or can have an azido group in place of a hydroxyl at the R¹ or R² position. In certain preferred embodiments, the NAD⁺ analogue comprises a chemical structure of NAD+analogue 2 (R¹=OH, R²=alkyne, e.g., propargyl) or NAD+analogue 6 (R¹=OH, R²=azido), also termed 3′-alkyne and 3′-azido, respectively, which can be referred to herein as the 3′-azido compound, which compounds have the following structures:

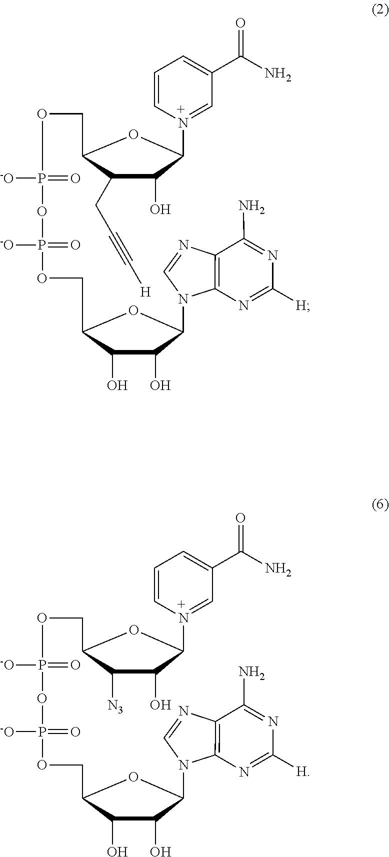

Accordingly, in some embodiments, a modified NAD⁺ analogue having a “click 5 chemistry” group—termed 3′-azido NAD⁺—is the NAD⁺ analogue illustrated below according to Formula II:

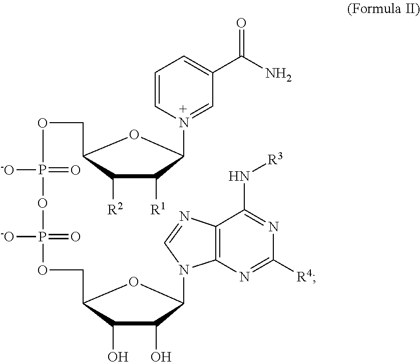

where R¹=OH, R²=N_{3,} R^{3 =}H, and R^{4 =}H.

In various embodiments, the ADPr polymers on the surface of the PARP protein incorporate the 3′-azido NAD+ moiety as a 3′-azido ADP-ribose having a structure according to Formula I:

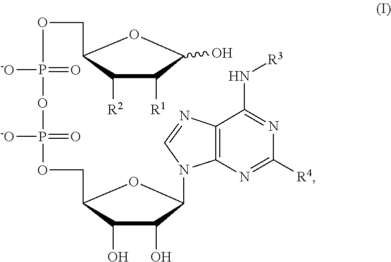

wherein R¹ is OH, R² is N_{3,} R³ is H, and R⁴ is H; i.e.,

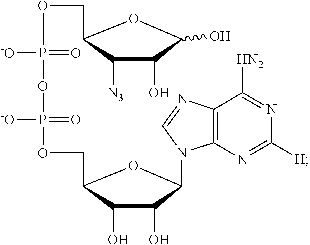

wherein an effector molecule is conjugated to at least one but less than all of the 3′-azido moieties. In some embodiments, the effector molecule is conjugated to a 3′-azido moiety of the ADPr polymers via a linker moiety.

### Antibody Molecules.

Various embodiments may include two or more antibodies conjugated to the modified ADPr polymers (e.g., 3′-azido) on the surface of the PARP protein, and preferably, the two antibodies are not the same (e.g., one antibody specifically binds to a first target antigen and a second antibody specifically binds to a second target antigen).

Techniques for preparing monoclonal antibodies against virtually any target antigen are well known in the art. See, for example, Köhler and Milstein, Nature 256: 495 (1975), and Coligan et al. (eds.), Current Protocols in Immunology, Vol. 1, pages 2.5.1-2.6.7 (John Wiley & Sons 1991). Briefly, monoclonal antibodies can be obtained by injecting mice with a composition comprising an antigen, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

MAbs can be isolated and purified from hybridoma cultures by a variety of well-established techniques. Such isolation techniques include affinity chromatography with Protein-A or Protein-G Sepharose, size-exclusion chromatography, and ion-exchange chromatography. See, for example, Coligan at pages 2.7.1-2.7.12 and pages 2.9.1-2.9.3. Also, see Baines et al., Methods in Molecular Biology, Vol. 10, pages 79-104 (The Humana Press, Inc. 1992).

After the initial raising of antibodies to the immunogen, the antibodies can be sequenced and subsequently prepared by recombinant techniques. Humanization and chimerization of murine antibodies and antibody fragments are well known to those skilled in the art, as discussed below.

The skilled artisan will realize that the claimed methods and compositions may utilize any of a wide variety of antibodies known in the art. Antibodies of use may be commercially obtained from a wide variety of known sources. For example, a variety of antibody secreting hybridoma lines are available from the American Type Culture Collection (ATCC, Manassas, Va.). A large number of antibodies against various disease targets, including but not limited to tumor-associated antigens, have been deposited at the ATCC and/or have published variable region sequences and are available for use in the claimed methods and compositions. (See, e.g., U.S. Pat. Nos. 7,312,318; 7,282,567; 7,151,164; 7,074,403; 7,060,802; 7,041,803; 6,949,244; 6,946,129; 6,943,020; 6,939,547; 6,921,645; 6,921,645; 6,921,533; 6,919,433; 6,919,078; 6,916,475; 6,905,681; 6,899,879; 6,893,625; 6,887,468; 6,887,466; 6,884,594; 66,187,287; 6,183,744; 6,129,914; 6,120,767; 6,096,289; 6,077,499; 5,922,302; 5,874,540; and 5,814,440, the Examples section of each of which is incorporated herein by reference.) These are exemplary only and a wide variety of other antibodies and their hybridomas are known in the art. The skilled artisan will realize that antibody sequences or antibody-secreting hybridomas against almost any disease-associated antigen may be obtained by a simple search of the ATCC, NCBI and/or USPTO databases for antibodies against a selected disease-associated target of interest. The antigen binding domains of the cloned antibodies may be amplified, excised, ligated into an expression vector, transfected into an adapted host cell and used for protein production, using standard techniques well known in the art. Isolated antibodies may be conjugated to therapeutic agents, such as camptothecins, using the techniques disclosed herein.

### Chimeric and Humanized Antibodies.

A chimeric antibody is a recombinant protein in which the variable regions of a human antibody have been replaced by the variable regions of, for example, a mouse antibody, including the complementarity-determining regions (CDRs) of the mouse antibody. Chimeric antibodies exhibit decreased immunogenicity and increased stability when administered to a subject. Methods for constructing chimeric antibodies are well known in the art (e.g., Leung et al., 1994, Hybridoma 13:469-476).

A chimeric monoclonal antibody may be humanized by transferring the mouse CDRs from the heavy and light variable chains of the mouse immunoglobulin into the corresponding variable domains of a human antibody. The mouse framework regions (FR) in the chimeric monoclonal antibody are also replaced with human FR sequences. To preserve the stability and antigen specificity of the humanized monoclonal, one or more human FR residues may be replaced by the mouse counterpart residues. Humanized monoclonal antibodies may be used for therapeutic treatment of subjects. Techniques for production of humanized monoclonal antibodies are well known in the art. (See, e.g., Jones et al., 1986, Nature, 321:522; Riechmann et al., Nature, 1988, 332:323; Verhoeyen et al., 1988, Science, 239:1534; Carter et al., 1992, Proc. Nat'l Acad. Sci. USA, 89:4285). In another embodiment, an antibody may be a human monoclonal antibody. Such antibodies may be obtained from transgenic mice that have been engineered to produce specific human antibodies in response to antigenic challenge.

### Human Antibodies.

Methods for producing fully human antibodies using either combinatorial approaches or transgenic animals transformed with human immunoglobulin loci are known in the art (e.g., Mancini et al., 2004, New Microbiol. 27:315-28; Conrad and Scheller, 2005, Comb. Chem. High Throughput Screen. 8:117-26. Such fully human antibodies are expected to exhibit even fewer side effects than chimeric or humanized antibodies and to function in vivo as essentially endogenous human antibodies. In certain embodiments, the claimed methods and procedures may utilize human antibodies produced by such techniques.

In one alternative, the phage display technique may be used to generate human antibodies (e.g., Dantas-Barbosa et al., 2005, Genet. Mol. Res. 4:126-40). Human antibodies may be generated from normal humans or from humans that exhibit a particular disease state, such as cancer (Dantas-Barbosa et al., 2005). The advantage to constructing human antibodies from a diseased individual is that the circulating antibody repertoire may be biased towards antibodies against disease-associated antigens.

In one non-limiting example of this methodology, Dantas-Barbosa et al. (2005) constructed a phage display library of human Fab antibody fragments from osteosarcoma patients. Generally, total RNA was obtained from circulating blood lymphocytes (Id.) Recombinant Fab were cloned from the u, y and K chain antibody repertoires and inserted into a phage display library (Id.) RNAs were converted to cDNAs and used to make Fab cDNA libraries using specific primers against the heavy and light chain immunoglobulin sequences (Marks et al., 1991, J. Mol. Biol. 222:581-97, incorporated herein by reference). Library construction was performed according to Andris-Widhopf et al. (2000, In: Phage Display Laboratory Manual, Barbas et al. (eds), 1^{st} edition, Cold Spring Harbor Laboratory Press, Cold Spring Harbor, N.Y. pp. 9.1 to 9.22, incorporated herein by reference). The final Fab fragments were digested with restriction endonucleases and inserted into the bacteriophage genome to make the phage display library. Such libraries may be screened by standard phage display methods. The skilled artisan will realize that this technique is exemplary only and any known method for making and screening human antibodies or antibody fragments by phage display may be utilized.

In another alternative, transgenic animals that have been genetically engineered to produce human antibodies may be used to generate antibodies against essentially any immunogenic target, using standard immunization protocols as discussed above. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A non-limiting example of such a system is the XENOMOUSE® (e.g., Green et al., 1999, J. Immunol. Methods 231:11-23, incorporated herein by reference) from Abgenix (Fremont, Calif.). In the XENOMOUSE® and similar animals, the mouse antibody genes have been inactivated and replaced by functional human antibody genes, while the remainder of the mouse immune system remains intact.

### Production of Antibody Fragments.

Some embodiments of the claimed methods and/or compositions may concern antibody fragments. Such antibody fragments may be obtained, for example, by pepsin or papain digestion of whole antibodies by conventional methods. For example, antibody fragments may be produced by enzymatic cleavage of antibodies with pepsin to provide a 5S fragment denoted F(ab′)₂. This fragment may be further cleaved using a thiol reducing agent and, optionally, a blocking group for the sulfhydryl groups resulting from cleavage of disulfide linkages, to produce 3.5S Fab′ monovalent fragments. Alternatively, an enzymatic cleavage using pepsin produces two monovalent Fab fragments and an Fc fragment. Exemplary methods for producing antibody fragments are disclosed in U.S. Pat. No. 4,036,945; U.S. Pat. No. 4,331,647; Nisonoff et al., 1960, Arch. Biochem. Biophys., 89:230; Porter, 1959, Biochem. J., 73:119; Edelman et al., 1967, Methods in Enzymology, page 422 (Academic Press), and Coligan et al. (eds.), 1991, Current Protocols in Immunology, (John Wiley & Sons).

Other methods of cleaving antibodies, such as separation of heavy chains to form monovalent light-heavy chain fragments, further cleavage of fragments or other enzymatic, chemical or genetic techniques also may be used, so long as the fragments bind to the antigen that is recognized by the intact antibody. For example, Fv fragments comprise an association of Vu and VI. chains. This association can be noncovalent, as described in Inbar et al., 1972, Proc. Nat'l. Acad. Sci. USA, 69:2659. Alternatively, the variable chains may be linked by an intermolecular disulfide bond or cross-linked by chemicals such as glutaraldehyde. See Sandhu, 1992, Crit. Rev. Biotech., 12:437.

Preferably, the Fv fragments comprise V_{H} and V_{L} chains connected by a peptide linker. These single-chain antigen binding proteins (scFv) are prepared by constructing a structural gene comprising DNA sequences encoding the V_{H} and V_{L} domains, connected by an oligonucleotides linker sequence. The structural gene is inserted into an expression vector that is subsequently introduced into a host cell, such as E. coli. The recombinant host cells synthesize a single polypeptide chain with a linker peptide bridging the two V domains. Methods for producing scFvs are well-known in the art. See Whitlow et al., 1991, Methods: A Companion to Methods in Enzymology 2:97; Bird et al., 1988, Science, 242:423; U.S. Pat. No. 4,946,778; and Pack et al., 1993, Bio Technology, 11:1271.

Another form of an antibody fragment is a single-domain antibody (dAb), sometimes referred to as a single chain antibody. Techniques for producing single-domain antibodies are well known in the art (see, e.g., Cossins et al., Protein Expression and Purification, 2007, 51:253-59; Shuntao et al., Molec Immunol 2006, 43:1912-19; Tanha et al., J. Biol. Chem. 2001, 276:24774-780). Other types of antibody fragments may comprise one or more complementarity-determining regions (CDRs). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See Larrick et al., 1991, Methods: A Companion to Methods in Enzymology 2:106; Ritter et al. (eds.), 1995, Monoclonal Antibodies: Production, Engineering and clinical Application, pages 166-179 (Cambridge University Press); Birch et al., (eds.), 1995, Monoclonal Antibodies: Principles and Applications, pages 137-185 (Wiley-Liss, Inc.).

### Antibody Variations.

In certain embodiments, the sequences of antibodies, such as the Fc portions of antibodies, may be varied to optimize the physiological characteristics of the conjugates, such as the half-life in serum. Methods of substituting amino acid sequences in proteins are widely known in the art, such as by site-directed mutagenesis (e.g. Sambrook et al., Molecular Cloning, A laboratory manual, 2^{nd} Ed, 1989). In preferred embodiments, the variation may involve the addition or removal of one or more glycosylation sites in the Fc sequence (e.g., U.S. Pat. No. 6,254,868, the Examples section of which is incorporated herein by reference). In other preferred embodiments, specific amino acid substitutions in the Fc sequence may be made (e.g., Hornick et al., 2000, J Nucl Med 41:355-62; Hinton et al., 2006, J Immunol 176:346-56; Petkova et al. 2006, Int Immunol 18:1759-69; U.S. Pat. No. 7,217,797; each incorporated herein by reference).

### Target Antigens and Exemplary Antibodies.

In a preferred embodiment, antibodies are used that recognize and/or bind to antigens that are expressed at high levels on target cells and that are expressed predominantly or exclusively on diseased cells versus normal tissues. Exemplary antibodies for use in embodiments of the invention include, but are not limited to, LL1 (anti-CD74), LL2 or RFB4 (anti-CD22), veltuzumab (hA20, anti-CD20), rituxumab (anti-CD20), obinutuzumab (GA101, anti-CD20), lambrolizumab (anti-PD-1 receptor), nivolumab (anti-PD-1 receptor), ipilimumab (anti-CTLA-4), RS7 (anti-epithelial glycoprotein-1 (EGP-1, also known as TROP-2)), PAM4 or KC4 (both anti-mucin), MN-14 (anti-carcinoembryonic antigen (CEA, also known as

CD66e or CEACAM5), MN-15 or MN-3 (anti-CEACAM6), Mu-9 (anti-colon-specific antigen-p), Immu 31 (an anti-alpha-fetoprotein), RI (anti-IGF-1R), A19 (anti-CD19), TAG-72 (e.g., CC49), Tn, J591 or HuJ591 (anti-PSMA (prostate-specific membrane antigen)), AB-PG1-XG1-026 (anti-PSMA dimer), D2/B (anti-PSMA), G250 (an anti-carbonic anhydrase IX MAb), L243 (anti-HLA-DR) alemtuzumab (anti-CD52), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (aka clivatuzumab, anti-mucin) and trastuzumab (anti-ErbB2/HER2). Such antibodies are known in the art (e.g., U.S. Patent Nos. 5,686,072; 5,874,540; 6,107,090; 6, 183, 744; 6,306,393; 6,653, 104; 6, 730.300; 6,899,864; 6,926,893; 6,962,702; 7,074,403; 7,230,084; 7,238,785; 7,238,786; 7,256,004; 7,282,567; 7,300,655; 7,312,318; 7,585,491; 7,612,180; and 7,642,239).

Other useful antigens that may be targeted using the described conjugates include carbonic anhydrase IX, B7, CCCL19, CCCL21, CSAp, HER-2/neu, BrE3, CD1, CDla, CD2, CD3, CD4, CD5, CD8, CD11A, CD14, CD15, CD16, CD18, CD19, CD20 (e.g., C2B8, hA20, 1F5 MAbs), CD21, CD22, CD23, CD25, CD29, CD30, CD32b, CD33, CD37, CD38, CD40,

CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM6, CTLA-4, alpha-fetoprotein (AFP), VEGF (e.g., AVASTIN®, fibronectin splice variant), ED-B fibronectin (e.g., L19), EGP-1 (TROP-2), EGP-2 (e.g., 17-1A), EGF receptor (ErbB1) (e.g., ERBITUX®), ErbB2, ErbB3, Factor H, FHL-1, Flt-3, folate receptor, Ga 733, GRO-β, HMGB-1, hypoxia inducible factor (HIF), HM1.24, HER-2/neu, insulin-like growth factor (ILGF), IFN-γ, IFN-α, IFN-β, IFN-k, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-IR, Ia, HM1.24, gangliosides, HCG, the HLA-DR antigen to which L243 binds, CD66 antigens, i.e., CD66a-d or a combination thereof, MAGE, mCRP, MCP-1, MIP-1A, MIP-1B, macrophage migration-inhibitory factor (MIF), MUC1, MUC2, MUC3, MUC4, MUC5ac, placental growth factor (PIGF), PSA (prostate-specific antigen), PSMA, PAM4 antigen, PD-1 receptor, NCA-95, NCA-90, A3, A33, Ep-CAM, KS-1, Le(y), mesothelin, 5100, tenascin, TAC, Tn antigen, Thomas-Friedenreich antigens, tumor necrosis antigens, tumor angiogenesis antigens, TNF-α, TRAIL receptor (R1 and R2), TROP-2, VEGFR, RANTES, T101, as well as cancer stem cell antigens, complement factors C3, C3a, C3b, C5a, C5, and an oncogene product. Antibodies to these and other targets may be generated using techniques that are well known in the art.

### Bispecific and Multispecific Antibodies.

Bispecific and multispecific antibodies refer to molecules designed to recognize two or more different epitopes or antigens. These antibodies may range from relatively small proteins, merely consisting of two linked antigen-binding fragments, to large immunoglobulin G (IgG)-like molecules with additional domains attached. Numerous methods to produce bispecific or multispecific antibodies are known, as disclosed, for example, in U.S. Patent No. 7,405,320, the Examples section of which is incorporated herein by reference. Bispecific antibodies can be produced by the quadroma method, which involves the fusion of two different hybridomas, each producing a monoclonal antibody recognizing a different antigenic site (Milstein et al., Nature, 1983; 305:537-540).

Another method for producing bispecific antibodies uses heterobifunctional cross-linkers to chemically tether two different monoclonal antibodies (Staerz, et al. Nature, 1985; 314:628-631; Perez, et al. Nature, 1985; 316:354-356). Bispecific antibodies can also be produced by reduction of each of two parental monoclonal antibodies to the respective half molecules, which are then mixed and allowed to reoxidize to obtain the hybrid structure (Staerz and Bevan. Proc Natl Acad Sci USA. 1986; 83:1453-1457). Another alternative involves chemically cross-linking two or three separately purified Fab′ fragments using appropriate linkers. (See, e.g., European Patent Application 0453082).

Other methods include improving the efficiency of generating hybrid hybridomas by gene transfer of distinct selectable markers via retrovirus-derived shuttle vectors into respective parental hybridomas, which are fused subsequently (DeMonte et al. Proc Natl Acad Sci USA. 1990, 87:2941-2945); or transfection of a hybridoma cell line with expression plasmids containing the heavy and light chain genes of a different antibody.

Cognate V_{H} and V_{L} domains can be joined with a peptide linker of appropriate composition and length (usually consisting of more than 12 amino acid residues) to form a single-chain Fv (scFv) with binding activity. Methods of manufacturing scFvs are disclosed, for example, in U.S. Pat. No. 4,946, 778 and U.S. Pat. No. 5,132,405. Reduction of the peptide linker length to less than 12 amino acid residues prevents pairing of Vand VI. domains on the same chain and forces pairing of V_{H} and V_{L} domains with complementary domains on other chains, resulting in the formation of functional multimers. Polypeptide chains of V_{H} and V_{L} domains that are joined with linkers between 3 and 12 amino acid residues form predominantly dimers (termed diabodies). With linkers between 0 and 2 amino acid residues, trimers (termed triabody) and tetramers (termed tetrabody) are favored, but the exact patterns of oligomerization appear to depend on the composition as well as the orientation of V-domains (V_{H}-linker-V_{L} or V_{L}- linker-V_{H}), in addition to the linker length.

These techniques for producing multispecific or bispecific antibodies exhibit various difficulties in terms of low yield, necessity for purification, low stability or the labor-intensiveness of the technique. More recently, a technique known as “dock and lock” (DNL) has been utilized to produce combinations of virtually any desired antibodies, antibody fragments and other effector molecules (see, e.g., U.S. Pat. Nos. 7,521,056; 7,527,787; 7,534,866; 7,550,143; 7,666,400; 7,858,070; 7,871,622; 7,906,121; 7,906,118; 8,163,291; 7,901,680; 7,981,398; 8,003,111 and 8,034,352). The technique utilizes complementary protein binding domains, referred to as anchoring domains (AD) and dimerization and docking domains (DDD), which bind to each other and allow the assembly of complex structures, ranging from dimers, trimers, tetramers, quintamers and hexamers. These form stable complexes in high yield without requirement for extensive purification. The DNL technique allows the assembly of monospecific, bispecific or multispecific antibodies. Any of the techniques known in the art for making bispecific or multispecific antibodies may be utilized in the practice of the presently claimed methods. (see, e.g., U.S. Pat. No. 9,446,123).

In various embodiments, a conjugate as disclosed herein may be part of a composite, multispecific antibody. Such antibodies may contain two or more different antigen binding sites, with differing specificities. The multispecific composite may bind to different epitopes of the same antigen, or alternatively may bind to two different antigens.

In one embodiment, the antibody is a monoclonal antibody (MAb). In other embodiments, the antibody may be a multivalent and/or multispecific MAb. The antibody may be a murine, chimeric, humanized, or human monoclonal antibody, and said antibody may be in intact, fragment (Fab, Fab′, F(ab)₂, F(ab′)₂), or sub-fragment (single-chain constructs) form, or of an IgG1, IgG2a, IgG3, IgG4, IgA isotype, or submolecules therefrom.

Bispecific and multispecific antibodies are useful in a number of biomedical applications. For instance, a bispecific or multispecific antibodies with binding sites for a tumor cell surface marker protein (i.e., antigen) and for a T-cell surface receptor can direct the lysis of specific tumor cells by T cells. For example, in some embodiments, In some embodiments, a bispecific or multispecific antibody specifically binds to an immune cell marker protein, such as a T-cell marker protein, comprising one or more of CD2, CD3, CD4, CD5, CD7, CD8, CD14, CD15, CD16, CD24, CD25, CD27, CD28, CD30, CD31, CD38, CD40L, CD45, CD56, CD68, CD91, CD114, CD163, CD206, LFA1, PD-1, ICOS, BTLA, KIR, CD137, OX40, LAG3, CTLA4, and a T-cell Receptor, and also to a cancer cell surface-marker protein comprising one or more of CLL-1, HER2, HER3, EGFR, CD33, CD34, CD38, CD123, TIM3, CD25, CD32, CD96, and protein death ligand 1 or 2 (PD-L1/L2).

In certain embodiments, one of the antibodies is Trastuzumab as disclosed in U.S. Pat. No. 5,821,337. Trastuzumab was the first monoclonal antibody developed for the treatment of HER2-positive breast cancer and has increased survival times for patients so that they are now the same as for patients with HER2-negative breast cancer.

### Linker Groups.

In certain embodiments, the linker may include a functional group or moiety that is capable of undergoing a click chemistry reaction. The click chemistry approach was originally conceived as a method to rapidly generate complex substances by joining small subunits together in a modular fashion. (See, e.g., Evans, RA, 2007, Aust J Chem 60(6):384-95.) Multiple variations of click chemistry reaction are known in the art, such as the Huisgen 1,3-dipolar cycloaddition copper catalyzed reaction (Tornoe et al., 2002, J. Org. Chem. 67:3057-64). Other alternative reaction mechanisms include cycloaddition reactions such as the Diels-Alder, nucleophilic substitution reactions (especially to small, strained rings like epoxy and aziridine compounds), carbonyl chemistry formation of urea compounds and reactions involving carbon-carbon double bonds, such as alkynes in thiol-yne reactions.

The azide alkyne Huisgen cycloaddition reaction uses a copper catalyst in the presence of a reducing agent to catalyze the reaction of a terminal alkyne group attached to a first molecule. In the presence of a second molecule comprising, for example, an azide moiety, the azide reacts with the activated alkyne to form a 1,4-disubstituted 1,2,3-triazole. Advantageously the copper catalyzed reaction occurs at room temperature and is sufficiently specific that purification of the reaction product is often not required (Tornoe et al., 2002, J. Org. Chem 67:3057). Advantageously, the azide and alkyne functional groups are largely inert towards biomolecules in aqueous medium, thus permitting the reaction to occur in complex solutions. The resultant triazole is chemically stable and may not be subject to enzymatic cleavage, making the click chemistry product highly stable in biological systems. And although the copper catalyst is toxic to living cells, the copper-based click chemistry reaction may be used in vitro for immunoconjugate formation.

A copper-free click reaction also has been proposed for covalent modification of biomolecules. (See, e.g., Agard et al., J. Am. Chem. Soc. 2004, 126, 46, 15046-15047). In this system, the copper-free reaction uses ring strain in place of the copper catalyst to promote a [3+2] azide-alkyne cycloaddition reaction. For example, cyclooctyne is an 8-carbon ring structure comprising an internal alkyne bond. The closed ring structure induces a substantial bond angle deformation of the acetylene, which is highly reactive with azide groups to form a triazole. Thus, cyclooctyne derivatives may be used for copper-free click reactions.

Another type of copper-free click reaction (Ning et al, Angew Chem Int Ed Engl. 2010 Apr. 12; 49(17): 3065-3068.) is based upon strain-promoted alkyne-nitrone cycloaddition. To address the slow rate of the original cyclooctyne reaction, electron-withdrawing groups are attached adjacent to the triple bond. Examples of such substituted cyclooctynes include difluorinated cyclooctynes, 4-dibenzocyclooctynol and azacyclooctyne. An alternative copper-free reaction involved strain-promoted alkyne-nitrone cycloaddition to give N-alkylated isoxazolines. The reaction was reported to have exceptionally fast reaction kinetics and was used in a one-pot three-step protocol for site-specific modification of peptides and proteins. Nitrones were prepared by the condensation of appropriate aldehydes with N-methylhydroxylamine and the cycloaddition reaction took place in a mixture of acetonitrile and water. These and other known click chemistry reactions may be used to attach carrier moieties to antibodies in vitro.

In some embodiments of the click chemistry reaction, the reactive group comprises an alkyne that is capable of undergoing a 1,3-cycloaddition reaction with an azide. Such suitable reactive groups include, but are not limited to, strained alkynes, e.g., those suitable for strain-promoted alkyne-azide cycloadditions (SPAAC), cycloalkynes, e.g., cyclooctynes, benzannulated alkynes, and alkynes capable of undergoing 1,3-cycloaddition reactions with azides in the absence of copper catalysts. Suitable alkynes also include, but are not limited to, substituted alkynes, e.g., fluorinated alkynes, aza-cycloalkynes, bicyclo[6.1.0]nonyne (BCN), and derivatives thereof. Linker-effector molecules comprising such reactive groups are useful for conjugating molecules that have been functionalized with azido groups.

In various embodiments, the linker includes a reaction group capable on undergoing a click chemistry reaction comprising an azide, alkyne, dibenzocyclooctyne, trans-cyclooctene, tetrazine, and bicyclo[6.1.0]nonyne. In one preferred embodiment, the linker is endo-BCN-PEG4-NHS ester. Commercial endo-BCN-PEG4-NHS ester linkers may be used to conjugate the BCN moieties rapidly on to the primary amines of an effector molecule. BCN groups are able to react with azide groups on an automodified PARP (e.g., PARP1) through copper free click chemistry, bridging the effector molecule together with automodified PARP (Leunissen et al., Chembioche, 2014 Jul. 7;15(10): 1446-51).

In certain embodiments, the solubility of the effector molecule may be enhanced by placing a defined polyethyleneglycol (PEG) moiety (i.e., a PEG containing a defined number of monomeric units) between the effector molecule and the ADPr polymer, wherein the defined PEG is a low molecular weight PEG, preferably containing 1-30 monomeric units, more preferably containing 1-12 monomeric units.

In some embodiments, the linker group is alkyne-PEG4-NHS ester linker having the structure:

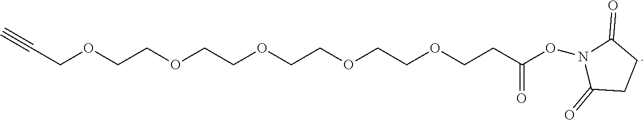 [SMILES: C#CCOCCOCCOCCOCCOCCC(=O)ON1C(=O)CCC1=O]

Importantly, while it is known in the art that “click chemistry” is an efficient chemical reaction that may be used to conjugate various moieties to a substrate, it is also well known in the art that even click chemistry reactions do not proceed to absolute 100% completion, regardless of the amount and type of reactants used. In line with this principle, the ADPr polymers will comprise at least one substituted dinucleotide (e.g., 3′-azido NAD⁺ moieties) with an unreacted azide moiety (i.e., that have not reacted with an antibody) in the final polymer structure.

Further, from a structural perspective, conjugating a large antibody molecule to a substituted dinucleotide of the ADPr polymers may preclude conjugation of another antibody molecule in the same region of the ADPr polymer (e.g., a region of the ADPr polymer having two consecutive substituted dinucleotide moieties) simply due to steric hinderance. Accessibility to the azido moieties of the substituted dinucleotides may be hindered further by the proximity of individual ADPr polymers to one another (e.g., polymers positioned adjacent to one another on the PARP protein may reduce or eliminate accessibility to the alkyne or azido moiety of the substituted dinucleotide in the area of the ADPr polymer in the closest proximity to the adjacent ADPr polymer).

In some embodiments, a poly ADP-ribose polymerase (PARP)-antibody conjugate comprises an automodified PARP comprising a plurality of poly ADP-ribose (ADPr) polymers, wherein the poly ADPr polymers comprise a plurality of 3′-azido ADP-ribose; and a first antibody molecule and a second antibody molecule conjugated to one or more of the plurality of 3′-azido ADP-ribose of the poly ADPr polymers, wherein the first antibody molecule specifically binds to a cancer cell surface marker protein and the second antibody molecule specifically binds to an immune cell surface marker protein, and wherein at least one of the plurality of 3′-azido ADP-ribose is not conjugated to either the first antibody molecule or the second antibody molecule (i.e., remains unconjugated). In some embodiments, the first antibody molecule and the second antibody molecule comprise a bispecific antibody wherein the first antibody molecule is a first antibody moiety of the bispecific antibody, and the second antibody molecule is a second antibody moiety of the bispecific antibody. In some embodiments, R¹ is O(CH₂)₂N₃, OH, or N₃; R² is OH or N₃; and R³ and R₄ are H; with the proviso that if R¹ is O(CH₂)₂N₃ or N₃, then R² is OH; or if R² is N₃, then R¹ is OH.

In some embodiments, the PARP-antibody conjugate comprises a first antibody and a second antibody that specifically binds separately to 1) an immune cell marker protein, such as a T-cell marker protein, comprising one or more of CD2, CD3, CD4, CD5, CD7, CD8, CD14, CD15, CD16, CD24, CD25, CD27, CD28, CD30, CD31, CD38, CD40L, CD45, CD56, CD68, CD91, CD114, CD163, CD206, LFAI, PD-1, ICOS, BTLA, KIR, CD137, OX40, LAG3, CTLA4, and a T-cell Receptor; and 2) a cancer cell surface-marker protein comprising one or more of CLL-1, HER2, HER3, EGFR, CD33, CD34, CD38, CD123, TIM3, CD25, CD32, CD96, and protein death ligand 1 or 2 (PD-L1/L2).

In some embodiments, a first antibody specifically binds to CD3 (CD3d:Entrez gene: 915; RefSeq: NP_000723.1, NP_001035741.1; CD3e: Entrez gene: 916; RefSeq: NP_000724.1; CD3g: Entrez gene: 917; RefSeq: NP_000064.1) and a second antibody specifically binds to HER2 (Entrez gene: 2064; RefSeq: NP_001005862.1, NP_001276865.1, NP_001276866.1).

In some embodiments, the antibody comprises a bispecific antibody comprising a first antibody moiety and second antibody moiety that specifically bind separately to 1) an immune cell marker protein, such as a T-cell marker protein, comprising one or more of CD2, CD3, CD4, CD5, CD7, CD8, CD14, CD15, CD16, CD24, CD25, CD27, CD28, CD30, CD31, CD38, CD40L, CD45, CD56, CD68, CD91, CD114, CD163, CD206, LFA1, PD-1, ICOS, BTLA, KIR, CD137, OX40, LAG3, CTLA4, and a T-cell Receptor; and 2) a cancer cell surface-marker protein comprising one or more of CLL-1, HER2, HER3, EGFR, CD33, CD34, CD38, CD123, TIM3, CD25, CD32, CD96, and protein death ligand 1 or 2 (PD-L1/L2).

In some embodiments, the antibody comprises a bispecific antibody comprising a first antibody moiety that specifically binds to CD3 (CD3d:Entrez gene: 915; RefSeq: NP_000723.1, NP_001035741.1; CD3e: Entrez gene: 916; RefSeq: NP_000724.1; CD3g: Entrez gene: 917; RefSeq: NP_000064.1) and a second antibody or second antibody moiety specifically binds to HER2 (Entrez gene: 2064; RefSeq: NP_001005862.1, NP_001276865.1, NP_001276866.1).

In some embodiments, the PARP-antibody conjugate comprises a plurality of antibodies and/or antibody moieties that specifically bind to an immune cell marker protein and/or a cancer cell surface-marker protein.

In some embodiments, a method of preparing a poly ADP-ribose polymerase (PARP)-antibody conjugate comprises combining a linker and an antibody molecule to provide an antibody-linker conjugate; combining a PARP and a substituted dinucleotide to provide an automodified PARP comprising a plurality of poly ADP-ribose (ADPr) groups on a surface of the automodified PARP, wherein the substituted dinucleotide comprises an azido group; and combining the automodified PARP and the antibody-linker conjugate under suitable conditions such that the antibody-linker conjugate is conjugated to the substituted dinucleotide through click chemistry to form the PARP-antibody conjugate. In some embodiments, the substituted dinucleotide comprises 3′-azido NAD of Formula II. In some embodiments, the plurality of ADP-ribose groups comprises a 3′azido ADP-ribose according to Formula I.

The disclosure also provides methods of treating cancer comprising administering an effective amount of any of the PARP-antibody conjugates described herein, or a composition thereof, to a subject in need thereof, wherein the PARP-antibody conjugate treats the cancer.

The term “cancer,” as used herein, refers to any benign or malignant abnormal growth of cells. Examples include, without limitation, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is selected from the group of tumor-forming cancers. In some embodiments, the cancer is breast cancer. In some embodiments, the breast cancer is a HER-2 positive breast cancer.

In one embodiment, a method for treating breast cancer comprises the steps of administering an effective amount of the PARP-antibody conjugate as described herein to a subject in need thereof, wherein the PARP-antibody conjugate treats the breast cancer. In another embodiment, a method for treating HER-2 positive breast cancer comprises the steps of administering an effective amount of the PARP-antibody conjugate as described herein to a subject in need thereof, wherein the PARP-antibody conjugate treats the HER-s positive breast cancer.

### Pharmaceutical Formulations

The compounds comprising the conjugates described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds comprising the conjugates described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds comprising the conjugates described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compounds (i.e., the PARP-antibody conjugate) may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Useful dosages of the compositions described herein can be determined by comparing their in vitro activity, and in vivo activity in animal models. Methods for the extrapolation of effective dosages in mice, and other animals, to humans are known to the art; for example, see U.S. Pat. No. 4,938,949. The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compounds comprising the conjugates described herein are conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound or composition as described herein formulated in such a unit dosage form.

The compounds comprising the conjugates described herein can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. PARP-Antibody Conjugates

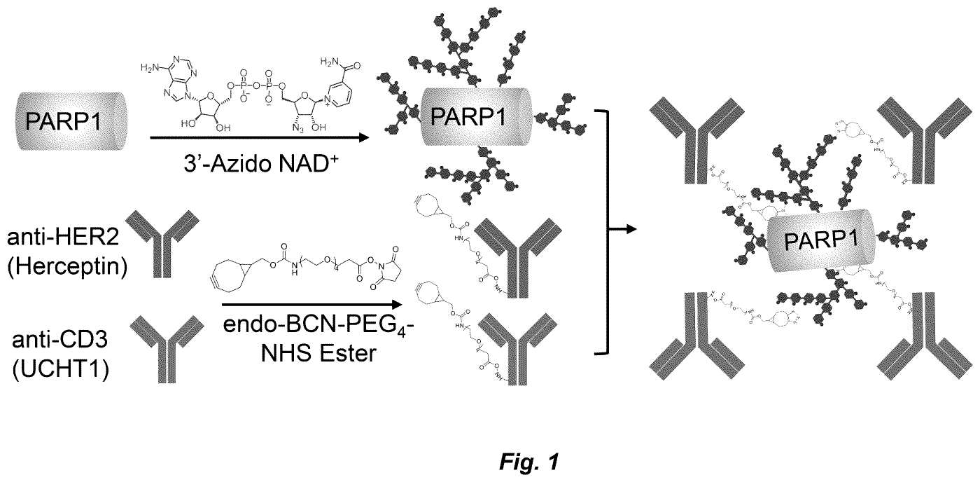

Post-translational PAR is a natural hydrophilic form of polymer that feature unique pharmacological properties. We envisioned that functionalized PAR polymers may enable facile conjugation of two different types of monoclonal antibodies for generating synthetic conjugates with immunotherapeutic potential. To explore this concept, we exploited recombinant full-length human PARP1 and the 3′-azido NAD⁺ molecule to produce functionalized PAR polymers. PARP1 catalyzes robust auto-modification upon activation by damaged DNA, resulting in PARylated PARP1. The 3′-azido substitution for the nicotinamide riboside moiety of NAD⁺ affords an analogue with high substrate activity for protein PARylation. Full-length monoclonal anti-human HER2 IgG and anti-human CD3 IgG functionalized with bicyclo[6.1.0]nonyne (BCN) were selected as model antibodies for conjugation with the 3′-azido PARylated PARP1 via copper-free click chemistry to generate a PAR polymer-based antibody (i.e., a bispecific antibody conjugate) (Shi et al., (2020) Chem Sci 11, 9303-9308) (FIG. 1).

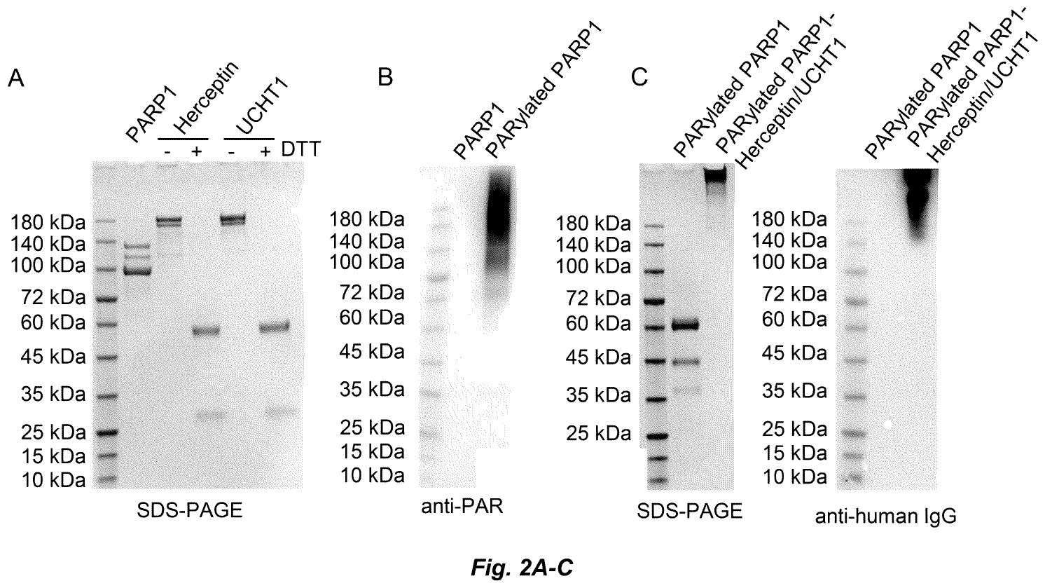

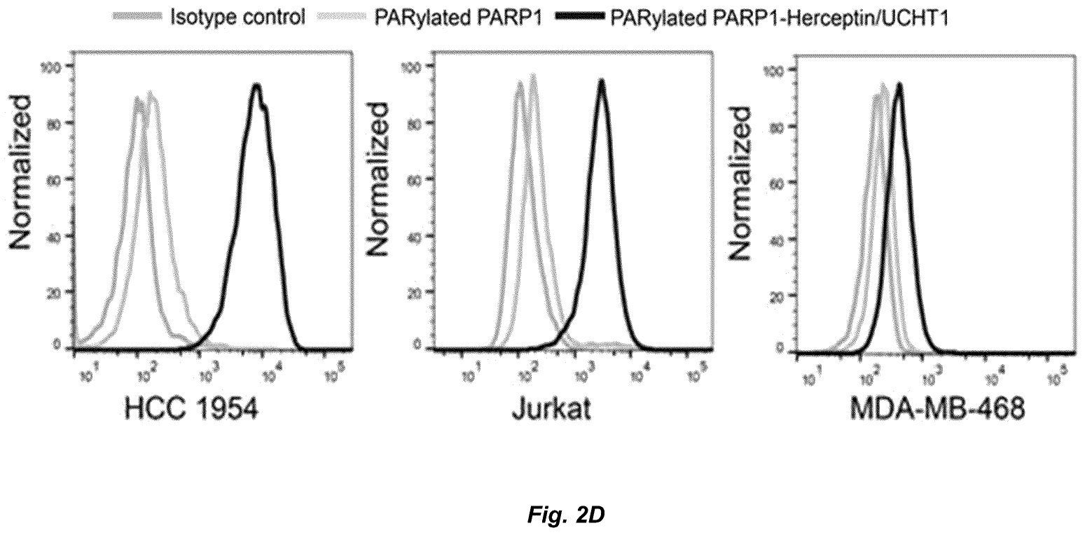

Full-length human PARP1 with a C-terminal His₆ tag was expressed and purified from bacteria using a previously established protocols (Zhang et al., (2022) Chem Sci 13, 1982-1991; Zhang et al., (2019) Nature communications 10, 4196). Coomassie-stained SDS-PAGE gels revealed both intact and cleaved human PARP1 following three-step chromatographic purification (FIG. 2A). The anti-human HER2 IgG (clone: trastuzumab; brand name: Herceptin) and anti-human CD3 IgG (clone: UCHT1) were transiently expressed in mammalian cells and purified through single-step affinity chromatography Dai et al., (2020) Biochemistry 59, 1420-1427; Zhang et al., (2015) J Am Chem Soc 137, 38-41; Cheng et al., (2018) J. Am. Chem. Soc. 140, 16413-16417; Shi et al., (2020) Molecular therapy : the journal of the American Society of Gene Therapy 28, 536-547). SDS-PAGE gels indicated that under the reducing condition, light and heavy chains of both Herceptin and UCHTI antibodies migrate at approximately 25 kDa and 50 kDa, respectively (FIG. 2A). Auto-modification of the purified human PARP1 with the 3′-azido NAD was then carried out. Immunoblot analysis using an anti-PAR monoclonal antibody showed strong smeared signals for the PARylated PARP1 but no signals for non-modified PARP1 (FIG. 2B), supporting generation of PAR polymers.

Following functionalization of the purified Herceptin and UCHTI IgGs with BCN groups (FIG. 1), 3′-azido PARylated PARP1 was incubated with the Herceptin-BCN and UCHT1-BCN at a molar ratio of 1:3:3 for three days at room temperature. The resulting PARylated PARP1-Herceptin/UCHT1 conjugate was then purified through size-exclusion chromatography. Coomassie-stained SDS-PAGE gels revealed that the PARylated PARP1-Herceptin/UCHT1 conjugate is characterized by molecular weights above 180 kDa (FIG. 2C). In comparison, the PARylated PARP1 features heterogeneous PAR polymers below the detection limit of Coomassie stain and three major cleaved fragments in a range of 35 to 65 kDa, likely due to high sensitivity to proteolysis during auto-PARylation. Furthermore, anti-human IgG-based immunoblot indicated that unlike PARylated PARP1 giving no signals, the PARylated PARP1-Herceptin/UCHT1 conjugate has significant levels of signal in regions over 140 kDa (FIG. 2C). These results support successful conjugation of IgG antibodies to the functionalized PAR polymers.

The binding specificity of the generated PARylated PARP1-Herceptin/UCHT1 conjugate toward HER2 and CD3 cognate antigens was examined by flow cytometry using HCC 1954 (HER²), Jurkat (CD³), and MDA-MB-468 (HER²⁻CD³⁻) cell lines (31,32). Flow cytometric analysis showed that the PARylated PARP1-Herceptin/UCHTI conjugate can not only bind to HCC 1954 cells but also Jurkat cells, whereas the PARylated PARP1 displays no significant binding to both cell lines (FIG. 2D). Both the PARylated PARP1-Herceptin/UCHT1 conjugate and PARylated PARP1 show little or no binding to MDA-MB-468 cells. This data indicates generation of the anti-human HER2/anti-human CD3 bispecific antibody conjugate through functionalized PAR polymer-mediated conjugation.

To further demonstrate dual targeting capability of the PARylated PARP1-Herceptin/UCHTI conjugate, confocal microscopy studies were performed for fluorescently labeled Jurkat cells (CD3) and breast cancer cells (HER²⁺ or HER²⁻) following incubation with the PARylated PARP1-Herceptin/UCHT1 conjugate. Confocal imaging analysis revealed crosslinking of HCC 1954 and Jurkat cells in the presence of the PARylated PARP1-Herceptin/UCHT1 conjugate (data not shown). As a control, PARylated PARP1 induces no formation of cell clusters for HCC 1954 and Jurkat cells. Moreover, the PARylated PARP1-Herceptin/UCHT1 conjugate gives rise to no crosslinking of MDA-MB-468 and Jurkat cells under the same conditions. The confocal microscopic results indicate that the PAR polymer-based bispecific antibody conjugate could engage CD3-expressing Jurkat cells to HER2-positive breast cancer cells through simultaneous binding to both cell-surface antigens.

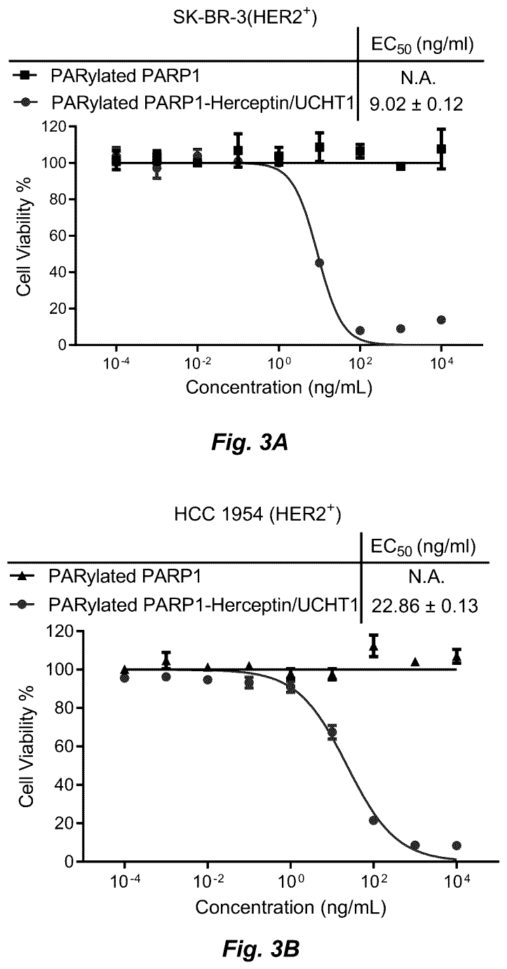

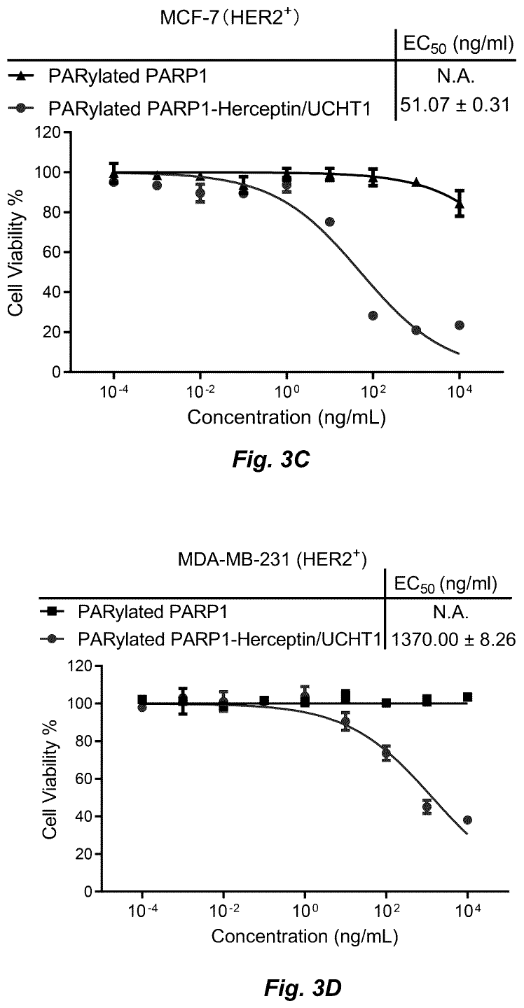

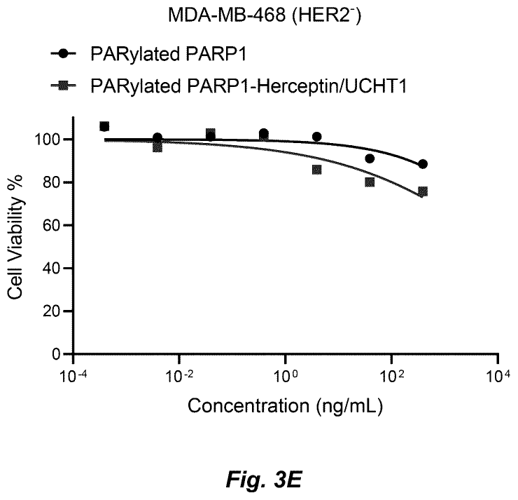

In vitro cytotoxicity of the PARylated PARP1-Herceptin/UCHTI conjugate was next evaluated using non-activated human PBMCs and breast cancer cell lines with varied levels of HER2 expression (Figure S1) (31,32). Following treatments of the mixtures of PBMCs (effector cells) and breast cancer cells (target cells) with various concentrations of the PARylated PARP1-Herceptin/UCHTI conjugate or PARylated PARP1, viabilities of target cells were measured by 3-(4,5-dimethylthiazol-2-yl)-2,5-diphenyltetrazolium bromide (MTT) assays (FIG. 3). Notably, the bispecific PARylated PARP1-Herceptin/UCHTI conjugate exhibits potent cytotoxicity (EC₅₀ of 9.02 ±0.12 ng mL⁻¹) against SK-BR-3 breast cancer cells in the presence of human PBMCs. Furthermore, the PARylated PARP1-Herceptin/UCHT1 conjugate could induce killing of HCC 1954 and MCF-7 cells by human PBMCs in an EC₅₀ of 22.86 and 51.07 ng mL^{-1,} respectively. In comparison, PARylated PARP1 shows little or no killing activity under the same conditions. The cytotoxicity of the PAR polymer-based bispecific antibody conjugate is correlated with HER expression levels of target cells. Unlike those breast cancer cells with high to moderate levels of HER2 expression, MDA-MB-231 and MDA-MB-468 cells with low or no HER2 expression (Data not shown) are less sensitive to the treatment of PARylated PARP1-Herceptin/UCHTI conjugate, supporting HER2-dependent cytotoxicity. These results indicate excellent in vitro potency and specificity for the PARylated PARP1-Herceptin/UCHT1 conjugate against HER2-positive breast cancer cells.

This study demonstrates facile synthesis of bispecific antibodies with functionalized PAR polymers. The resulting bispecific antibody conjugate displays marked cytotoxicity against HER2-expressing cancer cells in the presence of non-activated human PBMCs. In comparison with established genetic and chemical approaches for generating bispecific antibody conjugate, functionalized PAR polymers facilitate rapid conjugation of two or more types of monoclonal antibodies in different formats. Through this PAR polymer-based scaffold, bi- or multi-specific antibodies with distinct combinations could be readily created for functional assessment. Moreover, the polymeric PAR may increase avidity of conjugated antibodies for improved binding as well as allow conjugation of additional immune-modulating molecules for enhanced efficacy. Future studies include evaluation of in vivo pharmacological activities, development of new functionalized PAR polymers for orthogonal conjugation of distinct antibodies, and generation of different types of PAR polymer-based bi- and multi-specific antibodies for cancer immunotherapy.

In conclusion, an anti-human HER2/anti-human CD3 bispecific antibody conjugate was synthesized by utilizing functionalized PAR polymers. In the presence of human PBMCs, this bispecific antibody conjugate could induce potent cytotoxicity for HER2-positive breast cancer cells. The functionalized PAR polymers provide a valuable approach for generating bispecific antibody conjugate with immunotherapeutic potential.

### Example 2. Materials and Methods

Materials. Unless otherwise specified, all reagents were purchased from common commercial sources and used as received without further purification. Roswell Park Memorial Institute (RPMI) 1640 medium and Dulbecco's modified Eagle's medium (DMEM) were purchased from Corning Inc. Opti-modified Eagle's medium (Opti-MEM) and fetal bovine serum (FBS) were purchased from Thermo Fisher Scientific (Waltham, MA). BalanCD HEK293 medium and L-glutamine solution (200 mM) were purchased from FUJIFILM Irvine Scientific (Irvine, CA).

Cell lines. Breast cancer cell lines (SK-BR-3, HCC 1954, MDA-MB-231, and MDA-MB-468) and Jurkat cells were obtained from the American Type Culture Collection (ATCC) (Manassas, VA) and maintained in RPMI 1640 medium supplemented with 10% FBS at 37° C. and 5% CO₂. Breast cancer cell line MCF-7 was obtained from ATCC and cultured in DMEM medium with 10% FBS. Expi293F cells were purchased from Thermo Fisher Scientific and cultured in Expi293F expression medium with shaking at a speed of 125 rpm at 37° C. and 8% CO₂. Human PBMCs were obtained from HemaCare (Van Nuys, CA).

Chemical synthesis of 3′-azido NAD) . The 3′-azido NAD was synthesized according to a previously published method (Zhang et al., 2019. Nature communications 10, 4196).

Molecular cloning. pET-28a (+) vector encoding full-length human PARP1 with a C-terminal His6 tag. pFuse vectors encoding anti-HER2 antibody Herceptin heavy chain (HC) and light chain (LC) (pFuse-Herceptin HC and pFuse-Herceptin LC) were gifts.

Overlap extension polymerase chain reaction (PCR) was adopted to generate DNA fragments encoding the HC and LC of anti-human CD3 UCHTI antibody by exploiting variable regions of a previously constructed anti-human CD3 UCHTI scFv and constant regions of the Herceptin antibody. Primers used for the overlap extension PCRs include restriction enzyme sites of EcoRI and Nhel. Amplified fragments were ligated in-frame using T4 DNA ligase in a pFuse vector for the generation of mammalian expression constructs which were confirmed by DNA sequencing.

Protein expression and purification. The bacterial expression and purification of human full-length PARP1 were carried out (Shi et al., (2020) Chemical Science 11, 9303-9308;

Langelier et al., (2011) Methods Mol Biol 780, 209-226). The purified protein was further passed through an acrodisc unit with mustang E membrane (Pall Corporation, Port Washington, NY) through following the manufacturer's instructions. The final endotoxin levels (<0.5 EU mg-1 mL-1) were determined using Pierce LAL chromogenic endotoxin quantitation kits (Thermo Fisher Scientific). Purified PARP1 was analyzed by SDS-PAGE gels, flash frozen in liquid nitrogen, and stored at −80° C.

The anti-HER2 Herceptin antibody and anti-CD3 UCHTI antibody were expressed through transient transfection into Expi293F cells using polyethylenimine-Max (PEI-MAX) transfection regent (Polysciences, Warrington, PA) by following the manufacturer's instructions. Culture media of Expi293F cells transfected with the expression constructs were collected at day 3 and day 6 post-transfection and centrifuged at 4,000×g for 30 minutes. The supernatants were loaded on gravity flow columns packed with 2 mL of Protein G resin (GenScript, Piscataway, NJ), followed by washing with PBS. Antibodies was then eluted with elution buffer (100 mM glycine, pH 2.7), neutralized with 1 M Tris buffer (pH 8.0), dialyzed in PBS buffer for overnight and another 6 hours in PBS at 4° C., and concentrated using 30 kDa-cutoff amicon centrifugal concentrators (EMD Millipore Temecula, CA). Purified antibodies were analyzed by SDS-PAGE gels and stored at −80° C.

Antibody NHS-BON linker conjugation. A 20-fold molar excess of endo-BCN-PEG₄-NHS ester linker (BroadPharm, San Diego, CA; dissolved in 100% DMSO) was added into Herceptin or UCHT1 antibody in PBS, respectively. The solutions were mixed gently and allowed to react at room temperature for 2 hours. The reaction mixtures were then buffer exchanged to PBS with dilution factors over 1,000,000 using 30 kDa- cutoff amicon centrifugal concentrators to remove unreacted linkers.

PARP1 automodification. Purified human PARP1 (3 μM) was incubated with 150 μM of 3′-azido NAD⁺ or NAD⁺ in a reaction buffer containing 30 mM HEPES (pH 8.0), 5 mM MgCl₂, 5 mM CaCl_{2,} 250 mM NaCl, 1 mM DTT and 100 ng μL-⁻¹ activated DNA (Sigma-Aldrich, St. Louis, MO) at 30° C. for 8 hours. The reaction mixtures were then buffer exchanged to PBS using 30 kDa-cutoff amicon centrifugal concentrators.

Conjugation of antibodies with PARylated PARP1. Herceptin antibody-BCN and UCHT1 antibody-BCN were added into PARylated PARP1 solution with a molar ratio of 3:3:1. The conjugations were allowed to react for 3 days at room temperature. PARylated PARP1conjugates were purified through size-exclusion chromatography using a Superdex 200 Increase 10/300 GL column (GE Healthcare, Princeton, NJ) and eluted with PBS. The first peak eluted was collected and concentrated using amicon centrifugal concentrators with 30 kDa cutoff. Purified PARylated PARP1-Herceptin/UCHTI conjugates were examined by SDS-PAGE.

Immunoblot analysis. PARP1, PARylated PARP1, and PARylated PARP1-Herceptin/UCHT1 (2 μg of protein) were boiled with 10 mM DTT in NuPAGE LDS sample buffer (Thermo Fisher Scientific) at 98° C. for 5 minutes. Samples were then run on 4-20% ExpressPlus-PAGE gels (GenScript, Piscataway, NJ), transferred to immun-blot PVDF membranes (Bio-Rad Laboratories, Inc.). The membranes were subsequently blocked with 5% bovine serum albumin (BSA) in PBS with 0.1% Tween-20 (PBST) for 1 hour at room temperature, followed by incubation with anti-poly-ADP-ribose (PAR) monoclonal antibody (clone: 10 H, Santa Cruz Biotechnology) and anti-mouse IgG-HRP (catalog: 62-6520, Thermo Fisher Scientific) or anti-human IgG (H+L)-HRP (catalog: 5220-0277, SeraCare). The immunoblots were developed by additions of supersignal west pico PLUS chemiluminescent substrate (Thermo Fisher Scientific) and imaged with a ChemiDoc Touch Imaging System (Bio-Rad Laboratories, Inc).

Flow cytometric analysis. HER2 expression levels of SK-BR-3, HCC 1954, MCF-7, MDA-MB-231, and MDA-MB-468 cells were evaluated by flow cytometry. Cells were incubated with the Herceptin in PBS with 2% FBS at 4° C. for 30 minutes. Following three-time washing with PBS containing 2% FBS, cells were then incubated with Alexa Fluor-488 goat anti-human IgG (H+L) (catalog: A11013, Thermo Fisher Scientific) in PBS with 2% FBS at 4° C. for 30 minutes. After washing three times with PBS containing 2% FBS, cells were analyzed using a Fortessa X20 flow cytometer (BD Biosciences, San Jose, CA). Data were processed by FlowJo software (Tree Star Inc., Ashland, OR).

The binding of PARylated PARP1 and PARylated PARP1-Herceptin/UCHT1 conjugates to HER2 cell line HCC 1954, CD3 cell line Jurkat, and HER2- CD3- cell line MDA-MB-468 were evaluated by flow cytometry. PARylated PARP1 and PARylated PARP1-Herceptin/UCHT1 conjugates were first labeled with NHS-fluorescein (Thermo Fisher Scientific) at a 1:20 molar ratio according to the manufacturers' instructions. Free dyes were removed by buffer exchanged to PBS with dilution factors over 1,000,000 using 30 kDa-cutoff amicon centrifugal concentrators. Cells were incubated with fluorescein-labeled PARylated PARP1 or fluorescein-labeled PARylated PARP1-Herceptin/UCHT1 conjugates at 100 μg mL⁻¹ for 30 minutes at 4° C. and washed three times with PBS containing 2% FBS. Samples were analyzed using the Fortessa X20 flow cytometer and data were processed by FlowJo software.

Confocal microscopy of cell-cell crosslinking. HCC 1954 cells and MDA-MB-468 cells were stained with MitoSpy Red (BioLegend) and Jurkat cells were stained with CFSE (BioLegend) by following the manufacturer's instructions. Jurkat cells (6×10⁴) were incubated with PARylated PARP1 or PARylated PARP1-Herceptin/UCHTI conjugates (0.1 mg mL⁻¹) in 100 μL PBS for 30 minutes at 4° C. Following washing with 1 mL of cold PBS, Jurkat cells were resuspended in 500 μL RPMI-1640 medium with 10% FBS, then mixed with HCC1954 or MDA-MB-468 cells (2×104) in the same medium. The cell mixtures were subsequently added into clear bottoms of 24-well plates and incubated for 5 hours at 37° C. with 5% CO₂. The cells were then gently washed four times with PBS and imaged with a Leica SP8 confocal laser scanning microscope (Leica Microsystems Inc., Buffalo Grove, IL) equipped with a 40×, 1.3 NA PLAPO oil immersion objective lens using FITC (for CFSE) and rhodamine (for MitroSpy Red) filters. Images were processed using LAS X software (Leica Microsystems Inc., Buffalo Grove, IL).

In vitro cytotoxicity assays. Breast cancer cells (SK-BR-3, HCC 1954, MCF-7, MDA-MB-231, and MDA-MB-468) (1×10⁴ cells) were mixed with human PBMCs (effector cells) (1×10⁵ cells) and incubated for 40 hours in the presence of various concentrations of PARylated PARP1 or PARylated PARP1-Herceptin/UCHT1 conjugates at 37° C. Cells were then washed twice with PBS to remove PBMC suspensions, followed by additions of 3-(4,5-dimethylthiazol-2-yl)-2,5-diphenyltetrazolium bromide (MTT) solution. Following 3-hour incubation at 37° C. and subsequent additions of 100 μL of lysis buffer (20% SDS in 50% dimethylformamide, 0.5% (v:v) 80% acetic acid, 0.4% (v:v) 1 N HCl, pH 4.7), plates were incubated for 4 hours at 37° ° C. and measured for absorbance at 570 nm using a BioTek Synergy H1 Hybrid Multi-Mode Microplate reader (BioTek, Winooski, VT). Cell viability was calculated as: % cell viability=[(absorbanceₑₓₚₑᵣᵢₘₑₙₜₐₗ−absorbance_{spontaneous average)/(absorbance}_{maximal viability average}−absorbance_{spontaneous average})]×100.

### Example 3. Pharmaceutical Dosage Forms.

The following formulations illustrate representative pharmaceutical dosage forms that may be used for the therapeutic or prophylactic administration of a composition described herein, or a composition specifically disclosed herein (hereinafter referred to as ‘Composition X’ wherein the composition X comprises a PARP-antibody molecule conjugate as described herein):

| (i) Tablet 1 | mg/tablet |
|---|---|
| ‘Composition X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Composition X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Composition X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Composition X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Composition X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Composition X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Composition X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Composition X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Composition X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Composition X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Composition X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Composition X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference, and in particular, U.S. Pat. No. 11,497,787 to Zhang; U.S. Pat. No. 11,684,676 to Zhang et al.; and International Patent Publication No. WO2023/004424 to Zhang et al. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A poly ADP-ribose polymerase (PARP)-antibody conjugate comprising:
an automodified PARP comprising a plurality of poly ADP-ribose (ADPr) polymers, wherein the poly ADPr polymers comprise a plurality of 3′-azido ADP-ribose moieties; and
one or more antibody molecules conjugated to one or more of the plurality of 3′-azido ADP-ribose moieties, wherein the one or more antibody molecules specifically bind to both a cancer cell surface marker protein and an immune cell surface marker protein, and wherein at least one of the plurality of 3′-azido ADP-ribose moieties is not conjugated to the antibody molecules.

2. The PARP-antibody conjugate of claim 1 wherein the plurality of 3′-azido ADP-ribose moieties have a structure according to Formula I: 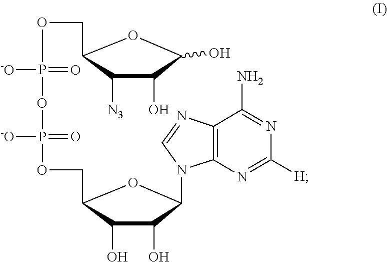
or a salt thereof; or a protonated moiety thereof.

3. The PARP-antibody conjugate of claim 1 wherein the automodified PARP is linked to the one or more antibody molecules through an alkyne-derived linking group, a polyethylene glycol linking group, or a combination there of.

4. The PARP-antibody conjugate of claim 3 wherein the alkyne-derived linking group is an alkyne-PEG₄-NHS ester linking group.

5. The PARP-antibody conjugate of claim 1 wherein the modified PARP is linked to the one or more antibody molecules through an alkyne-derived linking group, a cyclooctyne-derived linking group, or a polyethylene glycol linking group.

6. The PARP-antibody conjugate of claim 1 wherein the PARP is selected from the group consisting of PARP1, PARP2, PARP5a, and PARP5b.

7. The PARP-antibody conjugate of claim 6 wherein the PARP is PARP1.

8. The PARP-antibody conjugate of claim 1 wherein the one or more antibody molecules comprise a monoclonal antibody, polyclonal antibody, a single chain Fv, a bispecific antibody, a multispecific antibody, a Fv fragment, a Fab fragment, or a F(ab)2 fragment.

9. The PARP-antibody conjugate of claim 8 wherein the one or more antibody molecules comprises a bispecific antibody.

10. The PARP-antibody conjugate of claim 1 wherein the one or more antibody molecules comprise a first antibody and a second antibody, wherein the first antibody specifically binds to the immune cell surface marker protein and the second antibody specifically binds to the cancer cell surface marker protein.

11. The PARP-antibody conjugate of claim 1 wherein the immune cell surface marker protein comprises one or more of CD3, OX40, CD2, CD4, CD5, CD7, CD8, CD14, CD15, CD16, CD24, CD25, CD27, CD28, CD30, CD31, CD38, CD40L, CD45, CD56, CD68, CD91, CD114, CD163, CD206, LFA1, PD-1, ICOS, BTLA, KIR, CD137, LAG3, CTLA4, and a T-cell Receptor.

12. The PARP-antibody conjugate of claim 1 wherein cancer cell surface-marker protein comprises one or more of EGFR, CLL-1, HER2, HER3, CD33, CD34, CD38, CD123, TIM3, CD25, CD32, CD96, PD-L1, and PD-L2.

13. The PARP-antibody conjugate of claim 1 wherein the cancer cell surface-marker protein is HER2 and the immune cell surface-marker protein is CD3.

14. A composition comprising the PARP-antibody conjugate of claim 1 and a pharmaceutically acceptable carrier.

15. A method of preparing a poly ADP-ribose polymerase (PARP)-antibody conjugate comprising:
combining a linker and an antibody molecule to provide an antibody-linker conjugate;
combining a PARP and a plurality of an azido substituted dinucleotide to provide an automodified PARP comprising a plurality of poly ADP-ribose (ADPr) groups on a surface of the automodified PARP; and
combining the automodified PARP and the antibody-linker conjugate under suitable conditions such that the antibody-linker conjugate is conjugated to the azido substituted dinucleotide through click chemistry to form the PARP-antibody conjugate.

16. The method of claim 15 wherein the plurality of poly ADP-ribose (ADPr) groups have a structure according to Formula I: 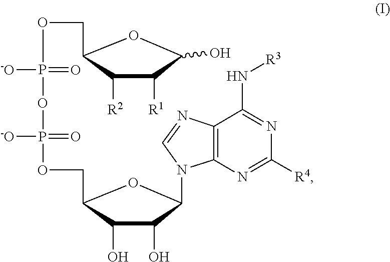
wherein R¹ is OH, R² is N₃, R³ is H, and R⁴ is H.

17. The method of claim 15 wherein the azido substituted dinucleotide is 3′-azido NAD+moieties have a structure according to Formula II: 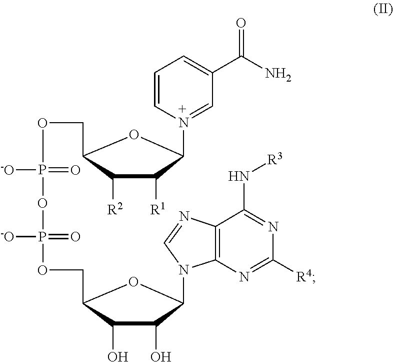
wherein R¹ is OH, R² is N₃, R³ is H, and R⁴ is H.

18. The method of claim 15 wherein the antibody molecule comprises a bispecific antibody, wherein the bispecific antibody specifically binds to both a cancer cell surface marker protein and an immune cell surface marker protein.

19. The method of claim 15 wherein the antibody molecule comprises a first antibody and a second antibody, wherein the first antibody specifically binds to a cancer cell surface marker protein and the second antibody specifically binds to an immune cell surface marker protein.

20. A method of treating cancer comprising:
administering an effective amount of the PARP-antibody conjugate of claim 1 to a subject in need thereof, wherein the PARP-antibody conjugate treats the cancer.
